(19) United States
(12) Patent Application Publication
(10) Pub. No.: US 2007/0073204 A1
(43) Pub. Date: Mar. 29, 2007

(54) ORTHOPEDIC BRACE WITH IMPROVED CLOSURE

(76) Inventors: Daniel SUAREZ, Lawrenceville, GA (US); Elian MARTINEZ, Dacula, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/533,865

(22) Filed: Sep. 21, 2006

Related U.S. Application Data
(60) Provisional application No. 60/720,117, filed on Sep. 23, 2005.

(51) Int. Cl. A61F 5/00

(52) U.S. Cl. 602/19; 602/5

(57) ABSTRACT

A lumbo-sacral orthopedic brace includes a rear member including a back portion and side portions. The brace also includes a front member. A closure is provided for adjustably and releasably securing the front member to the rear member and the closure includes straps coupled to the rear member. The straps are adapted to collectively span the front member and include hook and loop fastener elements to allow cooperating straps to be fastened to one another. The straps include finger loops into which the user or wearer can slip his or her fingers to conveniently manipulate the straps into position. In particular, the finger loops allow one to easily stretch out the straps to cinch the brace tight and to secure the straps to one another in a quick and easy manner.

### CROSS-REFERENCE TO RELATED APPLICATIONS

The present application claims the priority benefit of U.S. provisional patent application Ser. No. 60/720,117, filed Sep. 23, 2005, and incorporates the disclosure of the same herein by reference.

### TECHNICAL FIELD

The present invention relates to an orthopedic brace and in particular relates to an orthopedic brace having an improved closure to provide easy and quick attachment and adjustment.

### BACKGROUND OF THE INVENTION

A great many orthopedic braces exist for numerous ailments. One such orthopedic brace is a lumbo-sacral orthopedic brace. Typically, such braces can be comprised of rigid panels, soft panels, soft panels with rigid or semi rigid inserts, etc. In this general field, a great many designs can be found. Many such designs utilize straps for securing and adjusting the fit of the brace to the wearer.

For example, U.S. Pat. No. 5,967,998 discloses a lumbo-sacral orthopedic brace apparatus formed of flexible materials to fit over a patient's torso and has a flexible front section shaped to fit over the front of a patient's torso and covered with a hook and loop fastener loop material. A plurality of flexible straps are attached to the front section on either side thereof by being attached to a hook and loop fastener hook strap which is attached to the brace front section loop material. A flexible rear section is shaped to fit over the rear of a patient's torso and has a plurality of loops attached to each side thereof by being attached to a hook and loop hook strap which is attached to the rear section loop material. The straps on the flexible front section are looped through the loops on the rear section and are connected in groups to common flexible straps. Pulling one common strap pulls a plurality of straps through the loops. Each common strap has hook material attached thereto so that the common strap can be attached to the front section loop material in different positions and angles to adjust the stress of the rear flexible member on the rear of a patient's torso.

One problem with many such orthopedic braces is that the straps can be hard to operate and manipulate. In some instances, this problem is even more prolonged because many wearers of such a brace are experiencing some sort of physical difficulty of one sort or another at the time.

### SUMMARY OF THE INVENTION

Briefly described, in a first preferred form the present invention comprises a lumbo-sacral orthopedic brace and includes a rear member including a back portion and side portions. The brace also includes a front member. A closure is provided for adjustably and releasably securing the front member to the rear member. The closure includes straps coupled to the rear member. The straps are adapted to collectively span the front member and include hook and loop fastener elements to allow cooperating straps to be fastened to one another.

Preferably, the straps include finger loops into which the user or wearer can slip his or her fingers to conveniently manipulate the straps into position. In particular, the finger loops allow one to easily stretch out the straps to cinch the brace tight and to secure the straps to one another quickly and easily. Preferably, the finger loops are at one distal end of the straps. These first distal ends of the straps have some hook and loop material thereon, with some hook material on one side of the ends of the straps and some loop material on the other side. Preferably, to facilitate one strap overlying another and to be fastened thereto, the position of the finger loops on straps extending from one side of the rear member is different from the position of the finger loops on the straps extending from the other side of the rear member.

Preferably, the orthopedic brace includes loop material in locations to allow the ends of the straps to be temporarily “tacked” in place by temporarily securing the end of the strap to some loop material on the brace.

Preferably, the front member has a central panel and two smaller side panels. In one form, the side panels have loop material, while the central panel does not. In another form, the central panel includes loop material. Preferably, the distal ends of the straps bearing the finger loops have a width on the order of the width of a human hand, to facilitate manipulation of the straps by a user or wearer.

In one form, the straps are secured to the rear member using fixed loops. The fixed loops preferably are mounted securely to the rear member and the straps are threaded therethrough. Optionally, the rear panel or rear member can comprise a central back panel and two side panels.

Preferably, at least a portion of the straps comprises elastic material. Also preferably, a second, opposite end of the straps comprises a fastener panel. Preferably, the fastener panel includes loop material on one side and hook material on an opposite side.

Preferably, straps extending from one side of the rear member have loop material on the front, while straps extending from the other side of the rear member have hook material on the back.

In another preferred form the present invention comprises a lumbo-sacral orthopedic brace and includes a rear member including a back portion and side portions. The brace also includes a front member. A closure is provided for adjustably and releasably securing the front member to the rear member. The closure includes straps coupled to the rear member. The straps include finger loops into which the user or wearer can slip his or her fingers to manipulate the straps conveniently into position. In particular, the finger loops allow one to easily stretch out the straps to cinch the brace tight and to secure the straps to one another quickly and easily.

Preferably, the finger loops are at one distal end of the straps. These first distal ends of the straps have some hook and loop material thereon, with some hook material on one side of the ends of the strap and some loop material on the other side. Preferably, to facilitate one strap overlying another and to be fastened thereto, the position of the finger loops on straps extending from one side of the rear member is different from the position of the finger loops on the straps extending from the other side of the rear member.

Preferably, the straps are adapted to collectively span the front member and include hook and loop fastener elements to allow cooperating straps to be fastened to one another. In one form, the front member is provided without any loop material thereon so that the straps are fastened to one another without being secured to the front member. In an alternative form, the front member can be provided with loop material and then the straps can be secured directly thereto or can be secured to each other and also to the front member.

These and other features and advantages of the present invention will become more apparent upon reading the following specification in conjunction with the accompanying drawing figures.

### BRIEF DESCRIPTION OF THE DRAWING FIGURES

FIG. 1 is a perspective, partially disassembled view of a lumbo-sacral orthopedic brace according to a first preferred form of the invention.

FIG. 2A is a top view of the orthopedic brace of FIG. 1, shown in a closed position, such as when worn by a user.

FIG. 2B is a top view of the orthopedic brace of FIG. 1, shown in a partially opened position, such as when being donned by a user.

FIG. 3A is a front view of the end of one of the straps of the orthopedic brace of FIG. 1.

FIG. 3B is a back view of the strap end portion of the orthopedic brace of FIG. 3A.

FIG. 3C is a top view of the strap end portion of the orthopedic brace of FIG. 3A.

FIG. 3D is a side view of the strap end portion of the orthopedic brace of FIG. 3A.

FIG. 4A is a front view of the end of another one of the straps of the orthopedic brace of FIG. 1.

FIG. 4B is a back view of the strap end portion of the orthopedic brace of FIG. 4A.

FIG. 4C is a top view of the strap end portion of the orthopedic brace of FIG. 4A.

FIG. 4D is a side view of the strap end portion of the orthopedic brace of FIG. 4A.

FIG. 5 is a top view of end portions of strap portions of the orthopedic brace of FIG. 1, shown overlying and fastened to one another.

FIG. 6 is a top view of the orthopedic brace according to an alternative embodiment of the present invention and shown in a closed position, such as when worn by a user.

### DETAILED DESCRIPTION OF THE PREFERRED EMBODIMENTS

Referring now to the drawing figures, in which like reference numbers refer to like parts throughout the several views, preferred forms of the present invention will now be described by way of example embodiments. It is to be understood that the embodiments described and depicted herein are only selected examples of the many and various forms that the present invention may take, and that these examples are not intended to be exhaustive or limiting of the claimed invention. Also, as used in the specification including the appended claims, the singular forms “a,” “an,” and “the” include the plural unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

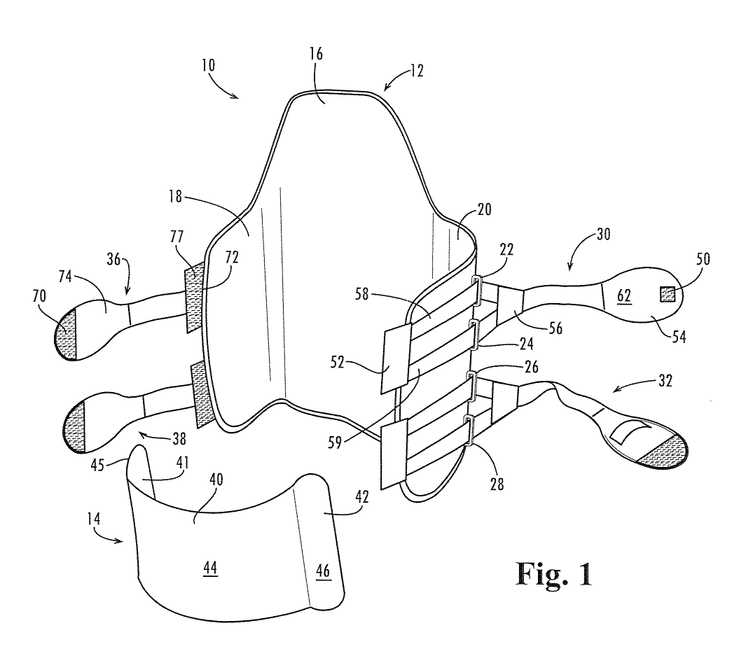

As shown in FIG. 1, a lumbo-sacral orthopedic brace 10 includes a posterior member 12 that can be releasably secured to an anterior member 14. The posterior member 12 has a back panel 16 for placement against a user's back and a pair of smaller sides 18 and 20 for wrapping around the hips of the user. The back panel 16 can be constructed of a rigid or a semi-rigid material, such as plastic, and covered with foam or fabric, or it can be constructed of a soft material with rigid or semi-rigid inserts. Preferably, the outer material for the posterior member comprises either loop material or a fabric having a weave that would cooperate with hook material. Optionally, the back panel 16 can have one or more pockets for receiving one or more inserts so as to provide additional lumbar support. Those skilled in the art will understand that the posterior member 12 can be made in any of a variety of sizes and shapes so as to accommodate users of various body sizes. Those skilled in the art will also understand that the posterior member 12 can also be constructed of various materials so as to provide lumbar and/or lumbosacral support.

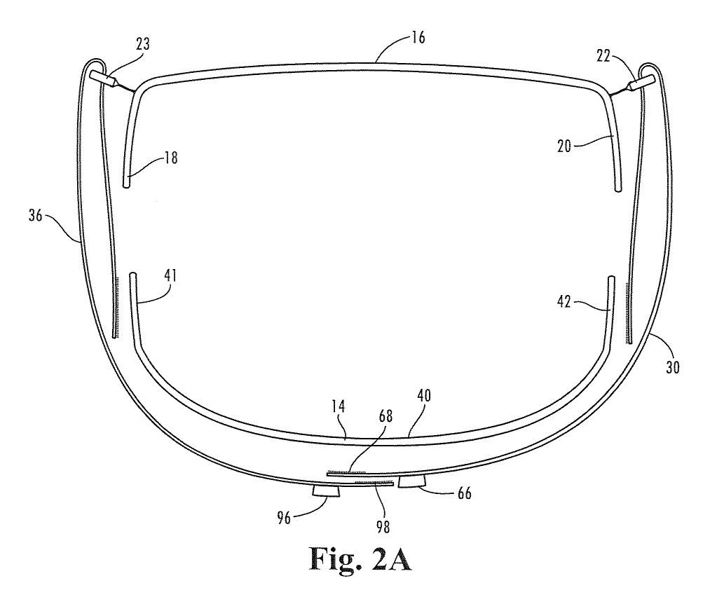

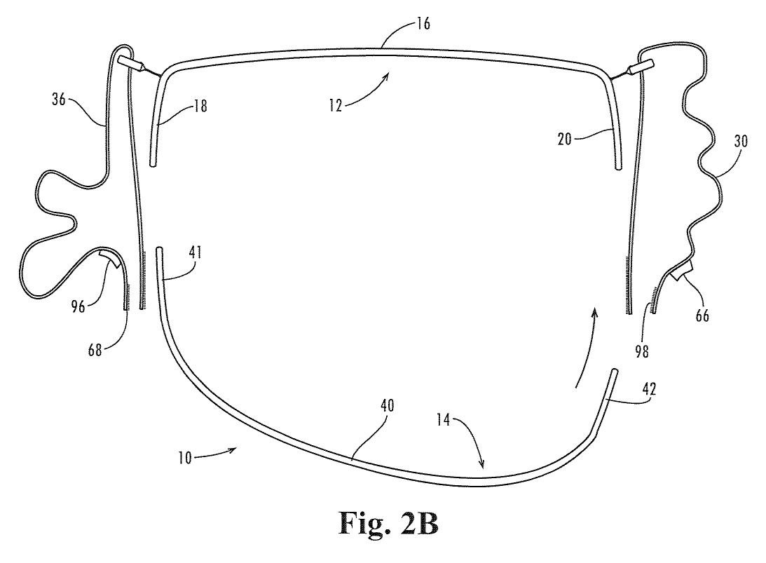

The side panel 20 includes a plurality of eyelets or belt loops 22, 24, 26, and 28 for capturing and retaining a pair of closure straps 30 and 32. Similarly, the side panel 18 includes four similar eyelets or belt loops (see for example eyelet 23, as shown in FIG. 2A), for capturing a pair of closure straps 36 and 38. As used herein, the term “eyelets” is used to describe the eyelets 22, 24, 26, and 28 for ease of description, and to minimize confusion even though the term “loop” may be more technically descriptive (because the term “loop” is also used to describe other features herein).

The anterior member 14 includes a front panel 40 and side panels 41 and 42. The anterior member 14 can be contoured so as to fit snugly against the abdomen of the user. The front panel 40 of the anterior member 14 has an outer surface 44, while the side panels 41 and 42 have outer surfaces 45 and 46, respectively. Preferably, the outer surfaces 45 and 46 of the side panels 41 and 42 are covered with loop material, while the outer surface 44 of the front panel is not. However, the outer surfaces 44, 45, and 46 can be covered with loop material or a fabric that is compatible with hook material (so that the hook material attaches to the fabric).

FIG. 2A shows a top view of the orthopedic brace 10 in a closed position. The straps 30 and 36 mate together so as to releasably connect the posterior member 12 to the anterior member 14, as shown in FIG. 2A. Thus, the straps are 30 and 36 are connected together, while straps 32 and 38 (although not shown in this view) are also secured together in a manner substantially similar or identical to that of the straps 30 and 36.

FIG. 2B shows a top view of the orthopedic brace 10 in a partially open position such as when being donned by the user. As depicted, the strap 30 and the strap 32 (although not shown) are connected to the posterior member 12 but are not connected to the anterior member 14, while the strap 36 and the strap 38 (although not shown) are connected to both the anterior member 14 and the posterior member. This configuration allows an opening for the user to slide the brace around his or her lower back. Alternatively, the straps 36 and 38 can be connected to only the posterior member 12, while the straps 30 and 32 are connected to both the anterior member 14 and the posterior member 12.

As described herein, the straps 30 and 32 are substantially similar to one another with the exception of their placement on the orthopedic brace, and thus only strap 30 will be described. The strap 30 has a distal broad strap end 50 and a base end 52. The broad strap end 50 has a bulb 54 permanently secured to an intermediate strap portion 56. Preferably, the distal broad strap end 50 has a width on the order of the width of a human hand to facilitate manipulation of the straps by a user or wearer. Between the intermediate strap portion 56 and the base end 52 is a pair of generally parallel elastic bands 58 and 59 that are permanently secured to both the base end and the intermediate portion. The eyelet 22 holds captive the elastic band 58, and the eyelet 24 holds captive the elastic band 59. Those skilled in the art will understand that the various portions of the strap 30 can be permanently secured together by stitching, gluing, bonding, or the like. Alternatively, the various portions can be semi-permanently or releasably connected together with various fasteners and/or fastening methods. Or, the straps can be a single unitary piece. In another embodiment, the strap 30 can include a single elastic band captured by a single eyelet, or the strap 30 can include a plurality of elastic bands captured by one or more eyelets.

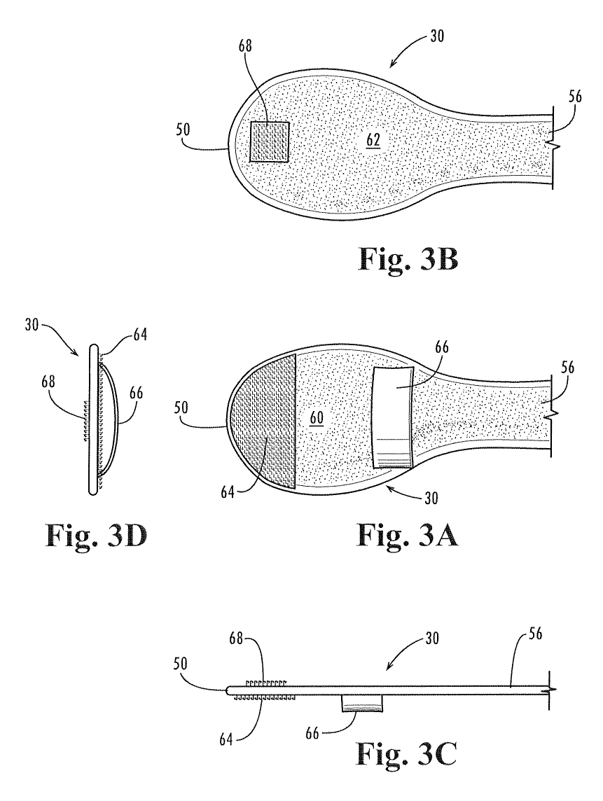

The strap 30 has an anterior surface 60 and a posterior surface 62, as more clearly shown in FIGS. 3A through 3D. The anterior surface 60 of the bulb 54 and the intermediate portion 56 comprise loop material, although a portion of the broad strap end 40 includes a semicircular patch 64 of hook material for connecting with a cooperating loop material portion of the strap 36. Those skilled in the art will understand that the patch 64 of hook material can include various shapes and sizes. The anterior surface of the bulb 54 includes a finger loop 66 permanently secured thereto through which the user can slip one or more fingers to conveniently manipulate the strap 30 into position. Preferably, the width of the finger loop 66 is on the order of the width of a human hand to facilitate the manipulation of the strap 30 by the user. Alternatively, the width of the finger loop 66 can be somewhat smaller, so as to accommodate one or more fingers of the user, or the finger loop 66 can be somewhat larger.

Additionally, the anterior surface 60 at the base portion 52 includes a strip (not shown in the figures) of hook material for releasably securing the strap to the side 20 of the orthopedic brace 10 or to the outer surface 46 of the side panel 42 of the anterior member 14. The posterior surface 62 of the strap 30 comprises mostly loop material with the exception of a small auxiliary patch 68 of hook material located proximate the distal broad end 50 for temporary tacking to the side panel 20 of the posterior member 12, or to the side panel 42 of the anterior member 14, or to the strap's base end 52.

The straps 36 and 38 are substantially similar to one another with the exception of their placement on the orthopedic brace 10, and thus only strap 36 will be described herein. The strap 36 has a distal a broad strap end 70 and a base end 72. The broad strap end 70 has a bulb 74 permanently secured to an intermediate strap portion 76. Preferably, the distal broad strap end 70 has a width on the order of the width of a human hand to facilitate manipulation of the straps by a user or wearer. Between the intermediate strap portion 76 and the base end 72 is a pair of generally parallel elastic bands (not shown) that are permanently secured to both the base end and the intermediate portion. The eyelet 23 holds captive the upper elastic band while a second eyelet (not shown) holds captive the lower elastic band, which is similar to the construction of the strap 30 with the elastic bands 58 and 59. Those skilled in the art will understand that the various portions of the strap 36 can be permanently secured together by stitching, gluing, bonding, or the like. Alternatively, the various portions can be semi-permanently or releasably connected together with various fasteners and/or fastening methods. Or, the strap can be a single unitary piece. In another embodiment, the strap 36 can include a single elastic band captured by a single eyelet, or the strap 36 can include a plurality of elastic bands captured by one or more eyelets.

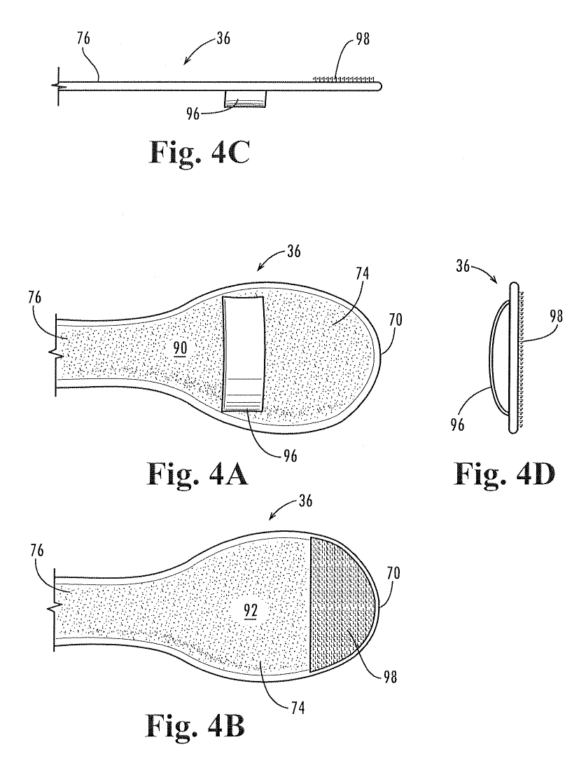

The strap 36 has an anterior surface 90 and a posterior surface 92, as more clearly shown in FIGS. 4A through 4D. The anterior surface 90 of the bulb 74 and the intermediate portion 76 comprise loop material. The anterior surface of the bulb 74 includes one finger loop 96 permanently secured thereto through which the user can slip one or more fingers to conveniently manipulate the strap 30 into position. Preferably, the width of the finger loop 96 is on the order of the width of a human hand to facilitate the manipulation of the strap 36 by the user. Alternatively, the width of the finger loop 96 can be somewhat smaller, so as to accommodate one or more fingers of the user, or the finger loop 96 can be somewhat larger.

Additionally, the anterior surface 90 at the base portion 72 includes a strip 77 of hook material for releasably securing the strap to the side 18 of the orthopedic brace 10. The posterior surface 92 of the strap 36 comprises mostly loop material with the exception of a semicircular patch 98 of hook material located proximate the distal broad end 70 for connecting with a cooperating, loop material portion of the strap 30 and for tacking to the side panel 18 of the posterior member 12, or to the side panel 41 of the anterior member 14, or to the strap's base end 72.

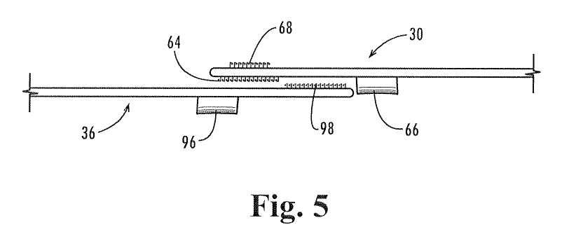

As shown in FIG. 5, the position of the finger loop 66 extending from one side of the posterior member 12 is different from the position of the finger loop 96 extending from the other side of the posterior member. Thus, when the strap 36 overlies and is secured to the strap 30, the finger loops 66 and 96, and consequently the user's hands, do not interfere with the closure of the straps 30 and 36.

Those skilled in the art will understand that even though the use of hook and loop material is described as preferable in the exemplary embodiments, other forms of fasteners can be used as well, including for example, fasteners that can be quickly released such as snaps, magnetic snaps, or other such fasteners. Those skilled in the art will also understand that fewer or additional straps can be used and still be within the scope of the present invention.

To use or wear the orthopedic brace 10, the user preferably configures the brace as shown in FIG. 2A and slides the open side around his or her lower back. The user slides his or her left hand (or a few fingers) into the finger grip 66 of the strap 30 and slides his or her right hand (or a few fingers) into the finger grip 96 of the strap 36. The user manipulates the straps, using the finger grips 66 and 96, to connect the straps such that the strap 36 overlies the strap 30, thereby forming a closure for the brace 10. The user secures the hook material 98 of the strap 36 to the loop material on the anterior surface 60 of the strap 30 while securing the hook material 64 of the strap 30 to the loop material of the posterior surface 92 of the strap 36, as shown in FIG. 2B. The straps 32 and 38 can be secured in a similar manner. Thus, as shown the straps 30, 32, 36, and 38 collectively span the front panel 40.

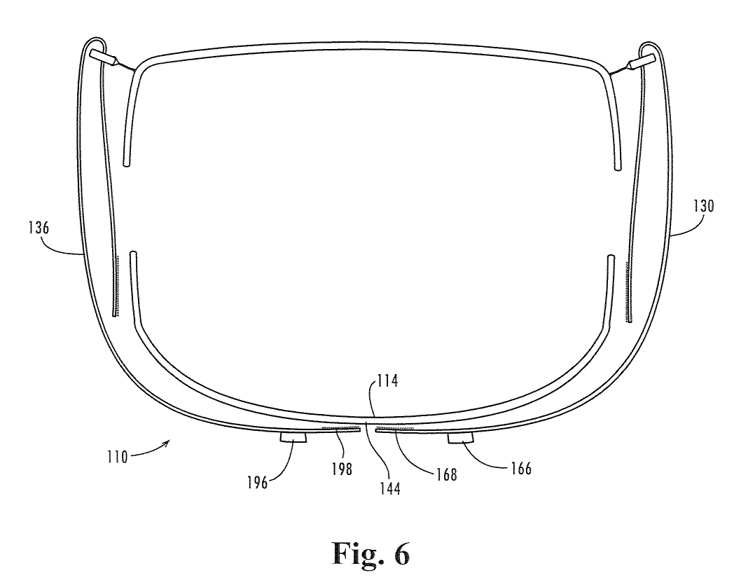

FIG. 6 shows a top view of an orthopedic brace 110 according to an alternative embodiment of the present invention. The orthopedic brace 110 is substantially similar to the orthopedic brace 10 but with the exceptions noted herein. The strap 136 does not overlie the strap 130, nor does the strap 138 overlie the strap 132. Rather, the straps 130 and 132 each include a patch of hook material 168 and 198 on the back (i.e., posterior) surfaces for connecting directly to the anterior surface 144 of the front panel 114 of the brace 110. Thus, preferably, the anterior surface 144 of the front panel 114 includes loop material for cooperating with the hook material of the patches on the straps. Alternatively, the anterior portion 114 can include a fabric having a weave such that the hook material can be secured thereto. Thus, the user inserts his or hands (or just a few fingers) into the finger straps 166 and 196 and attaches the straps directly to the front panel 140 of the anterior member 114, thereby forming a closure for the orthopedic brace 110.

While the invention has been described with reference to preferred and example embodiments, it will be understood by those skilled in the art that a variety of modifications, additions and deletions are within the scope of the invention, as defined by the following claims.

What is claimed is:

1. An orthopedic brace comprising:
a rear member including a back portion and side portions;
a front member; and
a closure for adjustably and releasably securing the front member to the rear member, the closure comprising straps coupled to the rear member and adapted to collectively span the front member, the straps comprising hook and loop fastener elements to allow cooperating straps to be fastened to one another.

2. The orthopedic brace as claimed in claim 1 wherein the straps include finger loops into which the user or wearer can slip one or more fingers to conveniently manipulate the straps into position.

3. The orthopedic brace as claimed in claim 2 wherein the finger loops are at a first distal end of the straps and the first distal ends of the straps have hook and loop material thereon, with some hook material on one side of the ends of the straps and some loop material on the other side.

4. The orthopedic brace as claimed in claim 3 wherein the position of the finger loops on the straps extending from one side of the rear member is different from the position of the finger loops on the straps extending from the other side of the rear member.

5. The orthopedic brace as claimed in claim 1 wherein the front member comprises a central panel and two smaller side panels.

6. The orthopedic brace as claimed in claim 5 wherein the side panels have loop material, while the central panel does not.

7. The orthopedic brace as claimed in claim 5 wherein the side panels and the central panel have loop material.

8. The orthopedic brace as claimed in claim 1 wherein the distal ends of the straps bearing the finger loops have a width on the order of the width of a human hand to facilitate manipulation of the straps by a user or wearer.

9. The orthopedic brace as claimed in claim 1 wherein the straps are secured to the rear member using fixed loops.

10. The orthopedic brace as claimed in claim 1 wherein a second, opposite end of the straps comprises a fastener panel comprising loop material on one side and hook material on an opposite side.

11. The orthopedic brace as claimed in claim 1 wherein straps extending from one side of the rear member have loop material on the front, while straps extending from the other side of the rear member have hook material on the back.

12. An orthopedic brace comprising:
a rear member including a back portion and side portions;
a front member; and
a closure for adjustably and releasably securing the front member to the rear member, the closure comprising straps coupled to the rear member and including finger loops into which the user or wearer can slip at least one of his or her fingers to conveniently manipulate the straps into position.

13. The orthopedic brace as claimed in claim 12 wherein the finger loops are at one distal end of the straps, the first distal ends of the straps have some hook and loop material thereon, with some hook material on one side of the ends of the strap and some loop material on the other side.

14. The orthopedic brace as claimed in claim 13 wherein to facilitate one strap overlying another and to be fastened thereto, the position of the finger loops on straps extending from one side of the rear member is different from the position of the finger loops on the straps extending from the other side of the rear member.

15. The orthopedic brace as claimed in claim 12 wherein the straps are adapted to collectively span the front member and include hook and loop fastener elements to allow cooperating straps to be fastened to one another.

16. The orthopedic brace as claimed in claim 12 wherein the front member is provided without any loop material thereon so that the straps are fastened to one another without being secured to the front member.

17. The orthopedic brace as claimed in claim 12 wherein the front member is provided with hook or loop material and then the straps can be secured directly thereto or can be secured to each other and also to the front member.

18. In a body brace adapted to be fitted around a user and having a plurality of straps for securing the brace around the user, the improvement therein comprising:
finger loops on the straps into which the user can slip at least one finger to manipulate the straps into position.

19. The improvement as claimed in claim 18 wherein to facilitate one strap overlying another and to be fastened thereto, the position of the finger loop on the strap extending from one side of the body brace is different from the position of the finger loop on the strap extending from the other side of the body brace.
